SEQUENCE LISTING (8 sequences)

SEQ ID NO: 1 - DNA, 21 bp, Artificial Sequence
  Description of Artificial Sequence hybridization probe (candida albicans)
aagtatggtgatgtattttca

SEQ ID NO: 2 - DNA, 19 bp, Artificial Sequence
  Description of Artificial Sequence hybridization probe (candida albicans)
aaactttcatcagtaacaa

SEQ ID NO: 3 - DNA, 23 bp, Artificial Sequence
  Description of Artificial Sequence hybridization probe (candida albicans)
tctccaggttatgctcatactag

SEQ ID NO: 4 - DNA, 23 bp, Artificial Sequence
  Description of Artificial Sequence hybridization probe (candida albicans)
aacaatcagaacactgaatcgaa

SEQ ID NO: 5 - DNA, 30 bp, Artificial Sequence
  Description of Artificial Sequence hybridization probe (candida albicans)
tcatgaatttgttttgaatgctaaattatc

SEQ ID NO: 6 - DNA, 28 bp, Artificial Sequence
  Description of Artificial Sequence hybridization probe (candida albicans)
ccagattaatggaaccaaaaaaatttgc

SEQ ID NO: 7 - DNA, 30 bp, Artificial Sequence
  Description of Artificial Sequence hybridization probe (candida albicans)
ccttatttaccatttagtggtggtagacat

SEQ ID NO: 8 - DNA, 32 bp, Artificial Sequence
  Description of Artificial Sequence hybridization probe (candida albicans)
ttaactacttttatttataatttaagatggac

(19) United States
(12) Patent Application Publication
(10) Pub. No.: US 2002/0098483 A1
(43) Pub. Date: Jul. 25, 2002

(54) Method for detecting resistant fungal cells in clinical material

(76) Inventors: Jurgen Loffler, Reutlingen (DE); Hermann Einsele, Tubingen (DE)

Correspondence Address: Cummings & Lockwood, Granite Square, 700 State Street, P.O. Box 1960, New Haven, CT, 06509-1960, US

(21) Appl. No.: 09/764,619

(22) Filed: Jan. 17, 2001

Related U.S. Application Data
(63) Continuation of application No. 09/284,155, filed on Jul. 14, 1999, now abandoned.
Application No. 09/284,155 is a 371 of international application No. PCT/EP97/05454, filed on Oct. 4, 1997.

(30) Foreign Application Priority Data
Oct. 22, 1996 (DE) 196 43 486.6

(51) Int. Cl. C12Q 1/68; C07H 21/04

(52) U.S. Cl. 435/6; 536/23.7

(57) ABSTRACT

A method is presented for detecting resistant fungal cells in clinical material. First, fungus-specific nucleic acids are extracted from clinical material. Then, the fungus-specific nucleic acids are hybridized with hybridization probes directed against nucleic acids segments of azole derivative-resistant fungal cells. Prior to the hybridization a PCR reaction may be performed in which segments of the 14-α-lanosterol demethylase gene are amplified. Primers and probes for the PCR rejection and the hybridization, respectively, are also presented.

### BACKGROUND OF THE INVENTION

1. Field of the Invention

The present invention relates to a method for detecting resistant fungal cells in clinical material.

The general interest in methods for detecting fungal cells must be considered in view of the fact that, especially in recent years, fungal species have acquired considerable importance as nosocomial pathogens in immunosuppressed patients.

2. Related Prior Art

The methods known hitherto for the analysis of fungal infections aim principally at making possible a diagnosis of the fungal infection and an identification of the pathogenic fungal species. This done by, for example, cultivation of fungal species from clinical material on suitable nutrient media, and possibly by the use of molecular biology methods.

Among the medically most significant facultatively pathogenic fungus genera is the genus Candida, belonging to the genus fungi imperfecti. It causes so-called Candida mycoses, also called candidoses. The most important pathogen within the Candida genus is the species Candida albicans, which causes not only infections of the skin and mucosa usually having a less serious course, but also deep organ mycoses or systemic mycoses. “Systemic mycoses” are understood to mean fungal infections which affect not only the skin or mucosa, but also further organs, organ systems, or indeed the entire organism. In the latter case the term “generalized” fungal infection is also used.

Detection of the pathogen agent in systemic mycoses is extraordinarily problematic, and in practice is often performed only post mortem. Molecular biology analytical methods have resulted in a substantial improvement here: they make it possible to detect fungal infections quickly and reliably, and to distinguish different fungal species from one another.

The publication “Rapid, polymerase chain reaction-based identification assays for Candida species” by Niesters et al. (1993), Journal of Clinical Microbiology, pp. 904-910, describes a method, based on the polymerase chain reaction (PCR), with which various Candida species can be detected and differentiated.

In this method, the fungus-specific nucleic acids are first extracted from clinical material and then further analyzed. Analysis is performed by detailed examination of the 18ssuRNA gene region. This is done by using suitable primers to amplify a segment of this gene region by PCR; the resulting PCR products are either sequenced, analyzed with the aid of restriction enzymes, or hybridized with specific hybridization probes. In the latter case, the hybridization probes are labeled by incorporating radioactive nucleotides, and detected by autoradiography. Different Candida species can then be distinguished from one another based on the sequence, the restriction pattern, or the hybridization pattern.

A further method with which Candida albicans infections can be diagnosed is described in the publication “Detection of surgical pathogens by in vitro DNA amplification. Part I. Rapid identification of Candida albicans by in vitro amplification of a fungus-specific gene” by Buchman et al. (1990), Surgery 108, pp. 338-347.

With this method, detection of Candida albicans infection is accomplished by PCR amplification of a different fungus-specific gene region, namely the gene for the enzyme 14-β-lanosterol demethylase.

The PCR products are detected in this case not by hybridization, but by separation in agarose gel and by staining the DNA with ethidium bromide.

In the two methods described above, molecular biology techniques are used to detect fungal infections in patient material and, as described in the publication of Niesters et al., additionally to distinguish different species of the genus Candida from one another.

Once the systemic fungal infection has been detected, it can be counteracted with various antimycotics, i.e. agents which inhibit the growth of fungi. The active ingredients for systemic use are azole derivatives, the polyene amphotericin B, flucytosine, griseofulvin, and terbinafine. The antimycotics ordinarily used are the azole derivatives, which include, for example, fluconazole.

Frequent use of this class of antimycotics has resulted very recently in the development of resistant fungal strains whose growth can no longer be inhibited with these therapeutic agents. It is not possible, however, to dispense entirely with the use of this agent, since in addition to its good effectiveness against systemic mycoses, in contrast to the other antimycotics it causes only mild and moreover harmless side effects. The essential problem in this context is that resistances of this kind cannot be detected early on, since no rapid tests are available for determining the presence of resistant fungal cells.

Azole derivatives are thus used initially as the “agent of choice”; only the fact that the patient shows no improvement despite high dosage and long-term treatment then allows the conclusion that he or she is infected with fungal cells that are resistant to azole derivatives. This diagnosis is often not made until the course of the infection in the patient takes a dramatic turn despite treatment with azole derivatives which, however, are ineffective in that particular case.

### SUMMARY OF THE INVENTION

In view of the above, it is an object of the present invention to create a rapid and reliable method with which azole derivative-resistant fungal cells can be specifically detected.

According to the present invention, this object is achieved by a method having the following steps:

a) Extraction of fungus-specific nucleic acids from clinical material; and

b) Hybridization of the fungus-specific nucleic acids with hybridization probes which are directed toward nucleic acid segments of azole derivative-resistant fungal cells.

By hybridizing fungus-specific nucleic acids using hybridization probes which specifically detect nucleic acid segments from azole derivative-resistant fungal cells, but not those from azole derivative-sensitive fungal cells, the detection of resistant fungal strains is now made accessible to a rapid, reproductible, and easy-to-perform molecular biology method. Detection can be thereby be accomplished quickly and on the basis of small quantities of clinical material. If a positive finding is made, i.e. if resistant fungal strains are present, a transition can be made to a different antimycotic agent with which the fungal infection is ultimately counteracted.

For this method, fungus-specific nucleic acids can preferably be extracted from blood, but also from biopsy material, sputum, mucosal swabs, or other patient material.

Either the fungus-specific DNA or the RNA can be isolated; it is then detected by DNA/DNA, DNA/RNA, or RNA/RNA hybridization.

It is possible to detect the hybridization in solution or on solid carriers, for example membranes or columns; the hybridization probes used are radioactively or nonradioactively labeled, and the specific hybridization is then detected by autoradiography or by enzyme-catalyzed color reactions.

The object of the invention is thereby completely achieved.

It is preferred, in the case of the method according to the present invention, if the hybridization probes are directed toward a DNA segment from the 14-β-lanosterol demethylase gene.

This is because the inventors of the present application have been able to demonstrate that in fungal species which exhibit resistance to azole derivatives, mutations occur in this gene region of the fungal DNA which, surprisingly, correlate in highly significant fashion with the clinical and microbiological finding of azole derivative resistance.

One possible explanation for this correlation is based on the recognition that the azole derivatives inhibit ergosterol synthesis in fungi. Ergosterol is a steroid which is embedded into the so-called plasmalemma, which is the phospholipid layer that adheres to the interior of the fungal cell wall. Ergosterol is synthesized in the cell from lanosterol, a precursor. The decisive step in the synthesis of ergosterol from lanosterol is catalyzed by the enzyme 14-α-lanosterol demethylase, abbreviated 14-DM.

Since ergosterol is an essential building-block of the plasmalemma, in the absence of ergosterol no cell division can take place. Continued synthesis of cell wall and plasmalemma is necessary for cell division.

Because the steroid ergosterol occurs specifically in fungi but not in human cells or bacteria, inhibition of ergosterol synthesis, which is essential for the growth of fungal cells, can be successfully used to counteract fungal infections.

It is thus an advantage of the method according to the present invention that hybridization probes are used against the gene which codes for the protein attacked directly by the azole derivatives. In resistant fungal cells, changes in the nucleic acid sequence occur frequently in this gene. The specific hybridization probes are then designed so that they detect these sequence changes, i.e. bind only to gene segments of fungal cells which exhibit resistance to azole derivatives.

It is further preferred in this method if the hybridization probes are directed toward a DNA segment from the 14-α-lanosterol demethylase gene of the species Candida albicans. In Candida albicans this gene is also called the ERG16 gene.

It is advantageous in this context that the hybridization probes make it possible to diagnose resistant strains of the most common pathogenic fungal species, namely Candida albicans.

In an improvement of the method according to the present invention, a PCR reaction in which segments of the 14-α-lanosterol demethylase gene are amplified is performed between steps a) and b).

This feature thus has the advantage that the method gains both sensitivity and specificity, since large quantities of starting material are generated which specifically contain only the required gene segment. The material amplified by PCR is then used, for example, in Southern hybridization.

In an improvement of the method according to the present invention, it is preferred if the nucleotide sequences SEQ ID no. 1 and SEQ ID no. 2 or the nucleotide sequences SEQ ID no. 3 and SEQ ID no. 4 are used in the PCR reaction as primer pairs.

It is advantageous in this context that, as the inventors have recognized, these primer pairs can be used to amplify DNA segments which contain DNA sequences characteristic of resistant fungal species. The resulting amplification products are much shorter than the entire gene, and thus allow for simpler subsequent processing.

In the method according to the present invention it is further preferred if one or more of nucleotide sequences SEQ ID nos. 5 through 8 of the enclosed Sequence Listing are used in step b) as hybridization probes.

This is because the inventors of the present application have recognized that, surprisingly, these hybridization probes can be used to distinguish resistant Candida species, whose resistance results in each case from only a single base exchange in the ERG16 gene, against sensitive strains which do not exhibit this mutation.

In an advantageous embodiment, in step b) the hybridization probes are labeled with digoxigenin and used in Southern hybridization. Detection of a specific hybridization is then accomplished by way of enzyme-conjugated anti-digoxigenin antibodies, the enzymes catalyzing color reactions.

The advantage here is that labeling of the hybridization probes does not need to be accomplished radioactively. In addition, large quantities of hybridizations probes can be labeled simultaneously; these can then be divided into aliquots and stored at −20° C., since they are stable for long periods. Aliquots from the same labeling reaction can then be thawed out and used for the individual detection reactions, thus guaranteeing good reproducibility over long periods of time.

Of course radioactive labeling methods and other nonradioactive labeling methods, for example labeling with biotin, are nevertheless also possible.

In Southern hybridization, it is advantageous that the nucleic acid to be analyzed can be rapidly applied onto a membrane, for example a microcellulose or nylon membrane. The quickest method in this context is the “blot-blot” or “dot-blot” method familiar to those skilled in the art.

It is also possible, however, first to separate out the DNA being analyzed in agarose gel, and only then to transfer it onto the membrane.

The fungal DNA can be used directly, or after prior PCR amplification, in Southern hybridization.

It is understood, however, that it is also possible to analyze fungus-specific RNAs. These can either be isolated directly from the cytoplasm of fungal cells, or can be produced by reverse transcription. Analysis can then be accomplished using Northern blot or other RNA detection reactions. Hybridization need not be accomplished on membranes, as for example in the case of Southern or Northern hybridization, but can also be performed in solution or on columns.

If hybridization probes having the nucleic acid sequences SEQ ID nos. 4 through 8 are used in step b), it is preferred if, after hybridization, a washing step is performed at a temperature which is approximately 1° C. less than the melting temperature (Tm) of the particular hybridization probe used.

The advantage here is that in this washing step, because of the relatively high temperature only those double-strand regions which do not exhibit any mispairing are stable. All paired hybridization probes whose DNA sequences do not completely match the corresponding fungal DNA segment are thus washed away in this washing step, and therefore no longer yield a signal in the detection reaction. In this fashion, a signal is obtained only if the fungal DNA derives from a resistant fungal cell which contains the mutation.

It is therefore possible in this manner, using the specific hybridization probes, to distinguish among gene segments which differ from one another by only a single base.

The invention further concerns the nucleotide sequences SEQ ID nos. 1 through 8 of the enclosed Sequence Listing.

It is preferred in this context if nucleotide sequences SEQ ID nos. 1 and 2 are used as primers for the PCR reaction, and nucleotide sequences SEQ ID nos. 5 and/or 6 as hybridization probes in the method for the detection of azole derivative-resistant fungal cells.

It is also preferred if nucleotide sequences SEQ ID nos. 3 and 4 are used as primers, and nucleotide sequences SEQ ID no. 7 or 8 as hybridization probes in the method according to the present invention.

The advantage of this feature is that the polymerase chain reaction thereby initially makes available, without difficulty, large quantities of easily handled DNA fragments comprising 300-400 base pairs; the base exchanges which may be present in those PCR fragments can then be identified using the hybridization probes.

Specifically, the inventors of the present application have succeeded in demonstrating that in azole derivative-resistant fungal strains, a number of individual base exchanges occur as compared with azole derivative-sensitive fungal strains. For example, a base exchange from T to G in the ERG16 gene causes amino acid phenylalanine No. 105 of enzyme 14-DM to mutate to a leucine. This T/G exchange can be detected on the gene level using the hybridization probe having nucleotide sequence SEQ ID no. 5. The inventors have also recognized that a base exchange from A to C can occur in resistant fungal strains, causing amino acid glutamine No. 142 to mutate to a proline. This mutation can be detected with the hybridization probe having nucleotide sequence SEQ ID no. 6. Two further base exchanges, both from G to A, have also been found. These cause glycine No. 464 of 14-DM to mutate to a serine, and valine No. 488 to mutate to an isoleucine. These two point mutations are detected with the hybridization probes having nucleotide sequences SEQ ID nos. 7 and 8, respectively.

Since resistant fungal strains contain either one or several of the base exchanges on the specifically amplified PCR fragment, it is advantageous if the corresponding hybridization probes are used simultaneously in Southern hybridization. A positive signal then indicates in all cases that a resistant fungal species is present. If hybridization is performed using only one of the hybridization probes, it is possible to detect which mutation has occurred in this resistant fungal strain, and whether one or several mutations are present.

It is understood, however, that nucleotide sequences SEQ ID no. 1 and SEQ ID no. 4 can also be combined as primers, so that the amplified PCR fragment of approximately 1,400 base pairs then contains all the mutations detectable with the hybridization probes having nucleotide sequences SEQ ID nos. 5 through 8.

The invention further concerns a kit for the analysis of fungal infections with azole derivative-resistant fungal strains, one or more of nucleotide sequences SEQ ID nos. 1 through 8 being contained in said kit.

A kit of this kind has the advantage that the method can be performed particularly quickly and easily, since the laboratory performing the method does not itself need to produce the primers and hybridization probes.

The kit can also contain all the solutions necessary for performing the polymerase chain reaction and hybridization. It is thereby possible for the method according to the present invention to be performed even in a routine laboratory by semi-skilled personnel. The method can moreover be performed quickly and without time-consuming preparations, and with high reproducibility, if all the necessary materials for many reactions are furnished in the kit.

Further advantages are evident from the description below.

It is understood that the features mentioned above and those yet to be explained below can be used not only in the respective combinations indicated, but also in other combinations or in isolation, without leaving the scope of the present invention.

### DETAILED DESCRIPTION OF PREFERRED EMBODIMENTS

Examples of performing of the individual steps of the method are given in the description below.

### EXAMPLE 1

### Cultivation of Candida albicans Strains

For analysis and for comparison between resistant and sensitive Candida albicans strains, patient material or yeast samples are incubated for 48 hours at 30° C. on Sabouraud glucose agar, a standard medium used for yeast culturing. Several colonies are then sampled, and placed in sterile 0.9% sodium chloride solution.

### EXAMPLE 2

### Disintegration of Fungal Cells and Isolation of Fungal DNA

The fungal cells are disintegrated by alkaline lysis (50 mM NaOH, 10 minutes, 95° C.) followed by neutralization and enzymatic treatment with Zymolase (Sigma). The proteins are denatured at 65° C. in Tris/EDTA and 10% SDS solution.

The resulting solution contains fungal cell debris as well as free fungal DNA, which now must be isolated.

This is done first by protein precipitation using 5 M potassium acetate, and precipitating the DNA by adding ice-cold isopropanol. The precipitation product is used for the subsequent process steps.

### EXAMPLE 3

### Amplifying a DNA Fragment from the ERG16 Gene

The purpose of the PCR reaction is to first amplify segments of the ERG16 gene to which the specific hybridization probes bind. The ERG16 gene, with a total length of 1,851 base pairs, is thereby subdivided into easily handled segments which can readily be amplified by PCR.

If DNA sequences SEQ ID nos. 5 and/or 6 are to be used as the hybridization probe, a PCR with primers having nucleotide sequence SEQ ID nos. 1 (upstream primer) and 2 (downstream primer) is performed.

The aforesaid primers then yield a PCR product which comprises the region from base 379 to base 676, i.e. approximately 300 base pairs, of the ERG16 gene.

If DNA sequences SEQ ID nos. 7 and/or 8 are to be used as hybridization probes, nucleotide sequences SEQ ID nos. 3 (upstream primer) and 4 (downstream primer) are used in the PCR.

These primers amplify the region from base 1360 to base 1774 of the ERG16 gene, i.e. a fragment of approximately 400 base pairs.

The PCR conditions are as follows:

| Buffer (50 ul): | |
|---|---|
| 10 mM Tris (pH 9.6) | |
| 50 mM NaCl | |
| 10 mM MgCl₂ | |
| 0.2 mg/ml BSA | |
| Polymerase | |
| 0.5 mM of each nucleotide | |
| 100 pM of each primer | |
| Initial denaturation | 3 min at 94° C. |
| Cycle denaturation | 0.5 min at 94° C. |
| Annealing: | 1 min at 62° C. |
| Extension: | 2 min at 72° C. |
| Terminal extension: | 5 min at 72° C. |
| No. of cycles: | 34 |

The high concentration of magnesium in the buffer ensures high specificity for the polymerase, which can operate in the extension step at its optimum temperature of 72° C.

The PCR reactions yield starting material in sufficiently large quantity so that further analysis can now be performed to determine whether the DNA derives from resistant or sensitive fungal cells.

The positions of the primers and the hybridization probes on the ERG16 gene are shown in Table I at the end of the Detailed Description.

### EXAMPLE 4

### Southern Hybridization of PCR Fragments

The PCR products obtained in Example 3 are thermally denatured and applied onto nylon membranes, for example using the slot-blot method familiar to those skilled in the art. The DNA is crosslinked on the nylon membrane. The hybridization probes are labeled by incorporating digoxigenin-labeled nucleotides using methods which are familiar to those skilled in the art (e.g. nick translation or random priming).

The DNA immobilized on the membrane is then initially denatured for 20 minutes with 0.4 N NaOH, and then neutralized with 2×SSPE (1×SSPE=150 mM NaCl, 10 mM sodium dihydrogen phosphate, 1 mM EDTA, pH 7.7). Prehybridization of the membrane is performed for 20 minutes in 6×SSPE, 5×Denhart's solution, 0.1% N-lauryl sarcosine sodium, 0.02% SDS at 42° C.

Hybridization is then performed in the aforementioned hybridization solution to which 30 pM digoxigeninated hybridization probe has been added, for 20 minutes at 42° C. The hybridization probes used are one or several of sequences SEQ ID nos. 5 through 8, individually, sequentially, or several concurrently.

Hybridization specificity is determined by washing steps which then follow. The first two washing steps are performed for 5 minutes at 42° C. in 2×SSPE, 0.1% SDS. Two washing steps are then performed for 7 minutes each in 6×SSPE, 1% SDS, the washing temperature being approximately, and preferably exactly, 1° C. less than the Tm value.

The melting temperatures of the hybridization probes, and the base exchanges detectable by way of the hybridization probes, are indicated in Table I.

If the nucleotide sequence of the hybridization probe does not exactly match the corresponding sequence of the PCR fragment, the hybridization probe is washed away in this step.

The detection reaction is then performed; this determines whether or not digoxigeninated hybridization probe is present on the membrane. This is done in a method according to the manufacturer's protocol of the Boehringer Mannheim company, using enzyme-conjugated anti-digoxigenin antibodies. The enzyme then catalyzes a reaction which causes creation of an insoluble color complex.

If one of the specific hybridization probes having the sequences SEQ ID nos. 5 through 8 has specifically hybridized with the fungal DNA from clinical material, the patient contains fungal cells which are resistant to azole derivatives. Treatment with azole-derivative antimycotics is therefore inadvisable for an infection with fungal cells of this kind, and a different treatment to counteract the Candida infection must be instituted.

TABLE I

| | | | Length | | | |
|---|---|---|---|---|---|---|
| | | | of | Length | | |
| | | Binding | nucleo- | of PCR | | |
| Nucleo- | | location | tide se- | frag- | | Base |
| tide se- | | on ERG16 | quence | ment | | ex- |
| quence | Type | gene (nt) | (nt) | (bp) | Tm (° C.) | change |
| SEQ ID | Up- | 379-400 | 21 | 297 | — | — |
| no. 1 | stream | | | | | |
| | primer | | | | | |
| 2 | Down- | 657-676 | 19 | | — | — |
| | stream | | | | | |
| | primer | | | | | |
| 3 | Up- | 1360-1383 | 23 | 414 | — | — |
| | stream | | | | | |
| | primer | | | | | |
| 4 | Down- | 1751-1774 | 23 | | — | — |
| | stream | | | | | |
| | primer | | | | | |
| 5 | Hybrid- | 448-747 | 30 | — | 74° C. | T −> G |
| | ization | | | | | |
| | probe | | | | | |
| 6 | Hybrid- | 557-584 | 28 | — | 74° C. | A −> C |
| | ization | | | | | |
| | probe | | | | | |
| 7 | Hybrid- | 1522-1551 | 30 | — | 82° C. | G −> A |
| | ization | | | | | |
| | probe | | | | | |
| 8 | Hybrid- | 1597-1628 | 32 | — | 76° C. | G −> A |
| | ization | | | | | |
| | probe | | | | | |

SEQUENCE LISTING

GENERAL INFORMATION:

APPLICANT:

NAME: Eberhard-Karls-Universität Tübingen, University Clinic

ADDRESS: Geissweg 3

CITY: Tübingen

COUNTRY: Germany

POSTAL CODE: 72076

TELEPHONE: (7071) 29-1

TELEFAX: (7071) 293966

DESCRIPTION OF THE INVENTION: Detection of Resistant Fungal Cells

NUMBER OF SEQUENCES:8

MACHINE-READABLE VERSION:

DATA MEDIUM: (to follow)

COMPUTER:

OPERATING SYSTEM:

SOFTWARE:

DATA FOR THIS APPLICATION:

ATTORNEY'S FILE NUMBER: 5402P132US

INFORMATION FOR SEQ ID NO. 1:

SEQUENCE CHARACTERISTICS:

LENGTH: 21 base pairs

TYPE: Nucleic acid

STRAND FORM: Single strand

TOPOLOGY: Linear

HYPOTHETICAL: YES

SEQUENCE DESCRIPTION: SEQ ID NO. 1:

AAGTATGGTG ATGTATTTTC A

INFORMATION FOR SEQ ID NO. 2:

SEQUENCE CHARACTERISTICS:

LENGTH: 19 base pairs

TYPE: Nucleic acid

STRAND FORM: Single strand

TOPOLOGY: Linear

HYPOTHETICAL: YES

SEQUENCE DESCRIPTION: SEQ ID NO. 2:

AAACTTTCAT CAGTAACAA

INFORMATION FOR SEQ ID NO. 3:

SEQUENCE CHARACTERISTICS:

LENGTH: 23 base pairs

TYPE: Nucleic acid

STRAND FORM: Single strand

TOPOLOGY: Linear

HYPOTHETICAL: YES

SEQUENCE DESCRIPTION: SEQ ID NO. 3:

TCTCCAGGTT ATGCTCATAC TAG

INFORMATION FOR SEQ ID NO. 4:

SEQUENCE CHARACTERISTICS:

LENGTH: 23 base pairs

TYPE: Nucleic acid

STRAND FORM: Single strand

TOPOLOGY: Linear

HYPOTHETICAL: YES

SEQUENCE DESCRIPTION: SEQ ID NO. 4:

AACAATCAGA ACACTGAATC GAA

INFORMATION FOR SEQ ID NO. 5:

SEQUENCE CHARACTERISTICS:

LENGTH: 30 base pairs

TYPE: Nucleic acid

STRAND FORM: Single strand

TOPOLOGY: Linear

HYPOTHETICAL: YES

SEQUENCE DESCRIPTION: SEQ ID NO. 5:

TCATGAATTT GTTTTGAATG CTAAATTATC

INFORMATION FOR SEQ ID NO. 6:

SEQUENCE CHARACTERISTICS:

LENGTH: 28 base pairs

TYPE: Nucleic acid

STRAND FORM: Single strand

TOPOLOGY: Linear

HYPOTHETICAL: YES

SEQUENCE DESCRIPTION: SEQ ID NO. 6:

CCAGATTAAT GGAACCAAAA AAATTTGC

INFORMATION FOR SEQ ID NO. 7:

SEQUENCE CHARACTERISTICS:

LENGTH: 30 base pairs

TYPE: Nucleic acid

STRAND FORM: Single strand

TOPOLOGY: Linear

HYPOTHETICAL: YES

SEQUENCE DESCRIPTION: SEQ ID NO. 7:

CCTTATTTAC CATTTAGTGG TGGTAGACAT

INFORMATION FOR SEQ ID NO. 8:

SEQUENCE CHARACTERISTICS:

LENGTH: 32 base pairs

TYPE: Nucleic acid

STRAND FORM: Single strand

TOPOLOGY: Linear

HYPOTHETICAL: YES

SEQUENCE DESCRIPTION: SEQ ID NO. 8:

TTAACTACTT TTATTTATAA TTTAAGATGG AC

Therefore, what I claim, is:

1. A method for detecting resistant fungal cells in clinical material, comprising the steps of:
a) extraction of fungus-specific nucleic acids from clinical material; and
b) hybridization of the fungus-specific nucleic acids with hybridization probes which are directed against nucleic acid segments of azole derivative-resistant fungal cells.

2. The method as in claim 1, wherein the hybridization probes are directed against a DNA segment from the 14-α-lanosterol demethylase gene.

3. The method as in claim 2, wherein the hybridization probes are directed against a DNA segment from the 14-α-lanosterol demethylase gene (ERG16 gene) of the species Candida albicans.

4. The method as in claim 2, wherein between steps a) and b) a PCR reaction is performed in which segments of the 14-α-lanosterol demethylase gene are amplified.

5. The method as in claim 3, wherein between steps a) and b) a PCR reaction is performed in which segments of the 14-α-lanosterol demethylase gene are amplified.

6. The method as in claim 4, wherein a primer for the PCR reaction is selected from the group consisting of SEQ ID-No.: 1, SEQ ID-No.: 2, SEQ ID-No.: 3 and SEQ ID-No.: 4.

7. The method as in claim 5, wherein a primer for the PCR reaction is selected from the group consisting of SEQ ID-No.: 1, SEQ ID-No.: 2, SEQ ID-No.: 3 and SEQ ID-No.: 4.

8. The method as in claim 1, wherein a hybridization probe for step b) is selected from the group consisting of SEQ ID-No.: 5, SEQ ID-No.: 6, SEQ ID-No.: 7 and SEQ ID-No.:8.

9. The method as in claim 4, wherein a hybridization probe for step b) is selected from the group consisting of SEQ ID-No.: 5, SEQ ID-No.: 6, SEQ ID-No.: 7 and SEQ ID-No.:8.

10. The method as in claim 6, wherein a hybridization probe for step b) is selected from the group consisting of SEQ ID-No.: 5, SEQ ID-No.: 6, SEQ ID-No.: 7 and SEQ ID-No.:8.

11. The method as in claim 1, wherein in step b) the hybridization probes are labeled with digoxigenin and used in Southern hybridization.

12. The method as in claim 8, wherein after hybridization, at least one washing step is performed at a temperature which is approximately 1° C. less than the melting temperature (Tm) of the particular hybridization probe used.

13. The method as in claim 9, wherein after hybridization, at least one washing step is performed at a temperature which is approximately 1° C. less than the melting temperature (Tm) of the particular hybridization probe used.

14. The method as in claim 10, wherein after hybridization, at least one washing step is performed at a temperature which is approximately 1° C. less than the melting temperature (Tm) of the particular hybridization probe used.

15. The nucleotide sequence SEQ ID no. 1 from the enclosed Sequence Listing.

16. The nucleotide sequence SEQ ID no. 2 from the enclosed Sequence Listing.

17. The nucleotide sequence SEQ ID no. 3 from the enclosed Sequence Listing.

18. The nucleotide sequence SEQ ID no. 4 from the enclosed Sequence Listing.

19. The nucleotide sequence SEQ ID no. 5 from the enclosed Sequence Listing.

20. The nucleotide sequence SEQ ID no. 6 from the enclosed Sequence Listing.

21. The nucleotide sequence SEQ ID no. 7 from the enclosed Sequence Listing.

22. The nucleotide sequence SEQ ID no. 8 from the enclosed Sequence Listing.

23. Use of the nucleotide sequences SEQ ID no. 1 and SEQ ID no. 2 as primers, and nucleotide sequences SEQ ID no. 5 and/or SEQ ID no. 6 as hybridization probes, in a method as in claim 4.

24. Use of the nucleotide sequences SEQ ID nos. 3 and SEQ ID no. 4 as primers, and nucleotide sequences SEQ ID no. 7 and/or SEQ ID no. 8 as hybridization probes, in a method as in claim 4.

25. A kit for the analysis of fungal infections with azole derivative-resistant fungal strains, containing at least one nucleotide sequences selected from the group consisting of SEQ ID-No.: 1, SEQ ID-No.: 2, SEQ ID-No.: 3, SEQ ID-No.: 4, SEQ ID-No.: 5, SEQ ID-No.: 6, SEQ ID-No.: 7, SEQ ID-No.: 8.

26. A kit for performing the method as in claim 1.
